(19) United States
(12) Patent Application Publication
(10) Pub. No.: US 2026/0272617 A1
(43) Pub. Date: Sep. 17, 2026

(54) Multi-Unit Bone Screw for Minimally Invasive Full-Arch Guided Implant Surgery

(71) Applicant: Amir Gadelmola, Dickinson, TX (US)

(72) Inventor: Amir Gadelmola, Dickinson, TX (US)

(21) Appl. No.: 19/386,223

(22) Filed: Nov. 11, 2025

Related U.S. Application Data
(60) Provisional application No. 63/718,989, filed on Nov. 11, 2024.

(51) Int. Cl. A61C 8/00; A61B 17/17; A61B 34/10

(52) U.S. Cl. CPC A61C 8/0095; A61B 17/176; A61B 34/10

(57) ABSTRACT

The embodiments herein are directed to a bone screw system. The bone screw system is configured for full-arch guided implant surgery. The bone screw system comprises a primary screw unit and a clamp attachment. The primary screw unit is configured for placement through soft tissue without extensive flapping. The clamp attachment unit that locks onto the primary screw, thereby forming a foundation for surgical guides.

### FIELD OF THE INVENTION

The present invention pertains to dental implantology, and specifically to systems and methods for performing full-arch guided implant surgeries, using a multi-unit bone screw system, known as the Guided Bone Screw (GBS), as a foundation for multi-piece surgical guides.

### BACKGROUND OF THE INVENTION

In traditional full-arch guided implant surgeries, the placement of custom-made metal or plastic bases is required to secure subsequent surgical guides. These bases are patient-specific, requiring extensive tissue flaps for proper seating and often resulting in significant bone loss and extended recovery times. The conventional techniques in said full-arched implant surgeries involve creating large tissue flaps to seat these base attachments, which are secured with anchor bone pins; and customized bases. This can be time-consuming, costly, and can lead to complications, such as increased recovery time and bone loss.

What is needed is the systems and methods herein directed to the Guided Bone Screw (GBS) to address the aforementioned technical problems. Stated another way, the GBS of the systems and methods herein obviates the need for large tissue flaps and custom-made bases.

### BRIEF SUMMARY OF THE INVENTION

The Guided Bone Screw (GBS) is a multi-unit bone screw system designed for minimally invasive full-arch guided implant surgeries. Unlike traditional solutions that require bulky base guides secured with bone screws or push pins, which leading to large tissue flaps, GBS introduces a streamlined approach, with a “one screw fits all” design. After the primary screw is inserted into the desired position in the bone through soft tissue, a clamp mechanism is attached. This clamp mechanism locks securely onto the screw head, serving two functions of: locking in place for stability and acting as the female attachment for subsequent steps of the surgical guide. The attachment allows for precise step-by-step guide seating throughout the procedure. The GBS eliminates the need for extensive tissue flaps, preserves bone, reduces recovery time, and supports multiple reuses of the screws after autoclaving until wear and tear affect performance.

In a variant, a bone screw system configured for full-arch guided implant surgery, comprises: a primary screw unit configured for placement through soft tissue without extensive flapping; and a clamp attachment unit that locks onto the primary screw, thereby forming a foundation for surgical guides.

In a variant, the surgical guides comprise a first teeth-supported guide, a second teeth-supported guide, a guide for bone reduction, and a guide for pilot drilling and osteotomy.

In a variant, the bone screw system further comprises a side tightening screw and a locking pin.

In a variant, a method of performing a full-arch guided implant surgery using the GBS comprises: performing digital planning, performing initial placement, performing scans, performing attachment placement, and performing subsequent surgical steps.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1 and FIG. 2 depicts a primary screw of the GBS of the systems and methods herein.

FIG. 3, FIG. 4, and FIG. 5 depict a clamp of the GBS of the systems and methods herein.

FIG. 6 and FIG. 7 depict a thread of the primary screw of the GBS of the systems and methods herein.

FIG. 8 depicts a three-dimensional (3D) rendering of the clamp of the GBS of the systems and methods herein.

FIG. 9 depicts a three-dimensional (3D) rendering of the clamp of the GBS of the systems and methods herein, which receives the clamp of the GBS of the systems and methods herein.

FIG. 10 and FIG. 11 depict a teeth-supported guide of the GBS of the systems and methods herein.

FIG. 12 depicts a screw inserted through a teeth-supported guide of the GBS of the systems and methods herein.

FIG. 13 depicts a teeth-supported guide for clamp placement of the GBS of the systems and methods herein.

FIG. 14 depicts a second clamp of the GBS of the systems and methods herein.

FIG. 15 depicts a first guide for bone reduction of the GBS of the systems and methods herein.

FIG. 16 depicts ensuring bone level via the GBS of the systems and methods herein.

FIG. 17 and FIG. 18 depicts subsequent guides for pilot drilling and osteotomy preparation.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

In the systems and methods herein, the GBS comprises a two-component bone screw system configured for use in full-arch guided implant surgeries. More particularly, the GBS comprises: primary screw unit (1) that is placed through soft tissue and anchored in the bone without the need for extensive flapping; and a secondary attachment unit, such as clamp attachment (2), that is subsequently secured to the screw head, thereby creating a stable foundation for subsequent surgical guides.

In the systems and methods, the Guided Bone Screw (GBS) is a multi-unit screw bone system that offers a significant technical advancement in the field of dental implantology, by providing a minimally invasive, cost-effective, and versatile solution for full-arch guided implant surgeries. By eliminating the need for custom-made bases and extensive tissue flaps, GBS of the systems and methods herein simplifies the surgical process and enhances patient outcomes.

In the systems and methods herein, the GBS eliminates the need for custom-made bases, by providing a universal solution that can be used in multiple patients. Additionally, the screws of the GBS, such as primary screw unit (1), can be reused until the connection weakens or the screw dulls, thereby making the GBS a cost-effective and efficient solution for full-arch implant surgeries.

In the systems and methods herein, the GBS is assembled and sold as a complete kit, including multiple screws, attachments, side screws, drills, and a screwdriver. Surgeons can maintain a stock of these screws, ready for any surgery, and order additional units as needed.

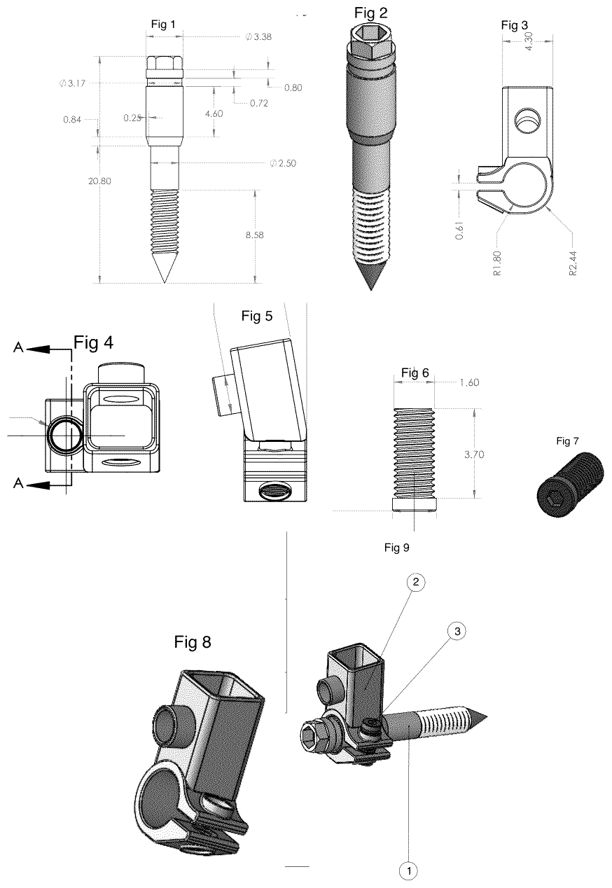

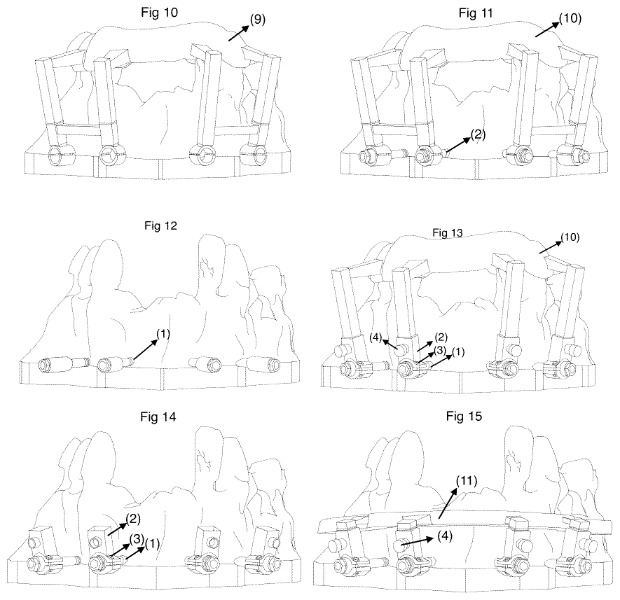

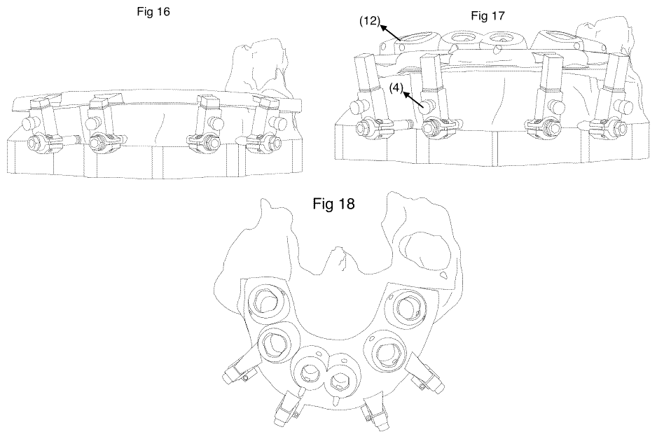

In the systems and methods herein, the screw and clamp attachment are depicted in FIG. 1, FIG. 2, FIG. 3, FIG. 4, FIG. 5, FIG. 6, FIG. 7, FIG. 8, and FIG. 9. In the systems and methods herein, the Guided Bone Screw (GBS), where a two-component bone screw system is depicted in an connected and assembled state (see FIG. 9), provides an innovative solution for minimally invasive, full-arch guided implant surgeries. More particularly. More particularly, the GBS comprises primary screw (1) and clamp attachment (2).

In the systems and methods herein, primary screw (1) is a specially designed screw (FIGS. 1 and 2) that is placed directly into the bone with the aid of a teeth-supported guide. The screw head is sandblasted, enhancing the surface of the screw head for accurate intraoral scanning. Primary screw (1) acts as the stable anchor point for the surgical process, reducing the need for tissue flaps. The threaded portion (see FIG. 1, FIG. 6, and FIG. 7) measures 8 mm in length and 2.5 mm in width, the screw head measures 3.5 mm in width and 6 mm in length. Clamp Attachment (2) is a clamp (see FIG. 3, FIG. 4, FIG. 5, and FIG. 8) that locks securely onto the screw head, using a teeth-supported guide (9) for precise placement. The clamp serves as the female attachment for step-by-step surgical guides, providing stability and precision throughout the surgery.

In the systems and methods herein, surgical steps that use the GBS comprise: pre-surgical planning (step 1); initial teeth-supported guide (9) and primary screw placement (step 2); intraoral scan after screw placement (step 3); teeth-supported guide (10) and clamp placement (step 4); step-by-step guide seating (step 5); implant placement (step 6); intraoral scan after implant placement (step 7); post-surgical closure (step 8); and screw reuse (step 9).

### Step 1. Pre-surgical Planning

A digital treatment plan is created using CBCT scans and intraoral scans or models.

The plan determines the precise locations for screw placement and guides the overall implant surgery process.

### Step 2. Initial Teeth-supported Guide (9) and Primary Screw Placement

The procedure begins with minimal tissue manipulation, such as small incisions or tissue punches at the planned insertion points; also primary screw (1) can be placed through soft tissue with no flap or tissue punch required. A teeth-supported guide (9), fabricated according to the pre-surgical plan, is seated in the patient's mouth (see FIGS. 10 and 11). This guide is designed to fit over the teeth and position the drill accurately for screw placement. Primary screw (1) of the GBS is inserted through the teeth-supported guide, passing through the soft tissue and into the bone at the pre-planned positions. The screw is placed (see FIG. 12) with precision using the guide to ensure correct depth and angulation. The screw head's sandblasted surface allows for easy intraoral scanning.

### Step 3. Intraoral Scan After Screw Placement

Once the primary screws are placed, an intraoral scan is performed to capture their exact position and angulation. This scan ensures that future steps, including the placement prosthesis, align perfectly, allowing for a passive fit in the temp restoration.

### Step 4 Teeth-Supported Guide (10) and Clamp Placement (See FIG. 13)

After the intraoral scan, a second teeth-supported guide, specifically designed for clamp placement, is used. This guide is seated over the patient's teeth, aligning with the already placed primary screw. Clamp attachment (2) is precisely positioned over the screw head (see FIG. 13) using this guide, thereby clamp attachment (2) is locked in place with a side-tightening screw (3) and locking pin (4). This ensures that clamp attachment (2) is securely attached without any deviation from the pre-surgical plan. The combination of screw and clamp (see FIG. 14) connections serves as the female connection for the subsequent surgical guides.

### Step 5. Step-by-step Guide Seating

With clamp attachment (2) secured (see FIG. 14), surgical guides are seated one by one onto clamp attachment (2), following a precise, stepwise process. For First Guide (11), the first guide (11) is used for bone reduction and placed onto the clamp attachment (2) secured with pin (4), thereby ensuring accurate bone leveling (see FIG. 16) as per the digital plan. For subsequent guides (see FIGS. 17 and 18), subsequent guides (12) for pilot drilling and osteotomy preparation are seated in sequence using the clamp as the anchor. Each guide is removed and replaced seamlessly, without disturbing the primary screw placement.

### Step 6. Implant Placement

Once subsequent guide (12) is seated, the implants are inserted with exact depth and angulation as planned, ensuring a stable and accurate implant placement.

### Step 7. Intraoral Scan After Implant Placement

After implant placement, a second intraoral scan is taken while primary screws (1) remain in position. This scan records the precise placement of the implants relative to the screws and helps ensure a passive fit for the final prosthesis. The accurate digital record minimizes the need for adjustments during the restorative phase.

### Step 8. Post-surgical Closure

After the implants are placed and the final scan is complete, the guides, clamps, and screws can be removed or left in place as needed. Since the initial tissue manipulation was minimal, only small sutures are required, promoting faster healing.

### Step 9. Screw Reuse

The screws and clamps in the GBS can be sterilized and reused across multiple surgeries. The screws and clamps in the GBS are durable and maintain performance until the connection weakens or the screw dulls, offering significant cost savings.

In a preferred embodiment of the GBS of the systems and methods herein, high-strength, biocompatible materials are used for the screw and attachment units. The screws of the GBS are designed to be reusable across multiple patients and procedures, offering a long-lasting and cost-effective solution for dental surgeons. The versatility allows the GBS for easy adaptation to various surgical scenarios, providing a reliable foundation for full-arch guided implant surgeries.

In alternative embodiment of the GBS of the systems and methods herein, there may be variations in the screw dimensions, attachment mechanisms, and materials used, provided that they remain within the scope of the invention's claims. These alternatives may be employed to address specific patient needs or surgical preferences.

Technical advantages of the GBS of the systems and methods herein are: minimal invasive surgery, precision, versatility, and cost-effectiveness.

With respect to minimally invasive surgery, the GBS of the system and methods herein reduce the need for extensive tissue flaps, preserving bone and shortening recovery time.

With respect to precision, the use of guides within the GBS of the systems and methods herein ensure precise placement of implants and attachments.

With respect to versatility, the clamp attachment mechanism of the GBS of the systems and methods herein is adaptable to various locking methods, providing flexibility in surgical procedures.

With respect to cost-effectiveness, the screws of the GBS of the systems and methods herein can be reused across multiple patients after autoclaving until the connection weakens or the screw dulls, reducing the need for custom-made bases.

The foregoing disclosure is not intended to limit the present invention to the precise forms or particular fields of use disclosed. It is contemplated that various alternative embodiments and/or modifications to the present invention, whether explicitly described or implied herein, are possible in light of the disclosure. Having thus described various example embodiments of the disclosure, persons of ordinary skill in the art will recognize that changes may be made in form and detail without departing from the scope of the invention. Thus, the invention is limited only by the claims.

What is claimed is:

1. A bone screw system configured for full-arch guided implant surgery, comprising:
a primary screw unit configured for placement through soft tissue without extensive flapping; and
a clamp attachment unit that locks onto the primary screw, thereby forming a foundation for surgical guides.

2. The bone screw system of claim 1, wherein the surgical guides comprise a first teeth-supported guide, a second teeth-supported guide, a guide for bone reduction, and a guide for pilot drilling and osteotomy.

3. The bone screw system of claim 1, further comprises a side tightening screw and a locking pin.

4. A method of performing a full-arch guided implant surgery using the GBS comprising:
performing digital planning,
performing initial placement,
performing scans,
performing attachment placement, and
performing subsequent surgical steps.
